(19) United States
(12) Patent Application Publication
(10) Pub. No.: US 2014/0045256 A1
(43) Pub. Date: Feb. 13, 2014

(54) CELL CHIP

(71) Applicant: SAMSUNG ELECTRO-MECHANICS CO., LTD., Suwon (KR)

(72) Inventors: Dong Woo LEE, Gyunggi-do (KR); Bo Sung KU, Gyunggi-do (KR); Sang Jin KIM, Gyunggi-do (KR); Moo-Yeal LEE, Gyunggi-do (KR)

(21) Appl. No.: 13/934,941

(22) Filed: Jul. 3, 2013

(30) Foreign Application Priority Data
Aug. 8, 2012 (KR) 10-2012-0086911

(51) Int. Cl. C12M 3/06

(52) U.S. Cl. CPC C12M 23/16
USPC 435/297.2; 435/299.1

(57) ABSTRACT

There is provided a cell chip including: a first substrate having biomaterials fixed thereto; a second substrate provided with one or more receiving space in which a culture medium is stored; and a circulation unit circulating the culture medium stored in the receiving space.

### CROSS-REFERENCE TO RELATED APPLICATIONS

This application claims the priority of Korean Patent Application No. 10-2012-0086911 filed on Aug. 8, 2012, in the Korean Intellectual Property Office, the disclosure of which is incorporated herein by reference.

### BACKGROUND OF THE INVENTION

1. Field of the Invention

The present invention relates to a cell chip, and more particularly, to a cell chip having a culture medium circulation structure.

2. Description of the Related Art

Demand for biomedical apparatuses and biotechnology for the rapid diagnosis of various human diseases has recently increased. Therefore, the development of an experimental apparatus and a device capable of rapidly providing diagnosis results for specific diseases, the diagnosis of which previously having taken a relatively long period of time in a hospital or a laboratory, has been actively conducted.

Meanwhile, in order to develop new medicines and determine the experimental stability thereof, it is necessary to culture an experiment subject (for example, a biomaterial including a cell). The biomaterial is generally cultured in a culture vessel or a culture dish in which a culture medium is stored.

Here, the culture medium of the culture dish may be changed in terms of the quality thereof, or may no longer react with the biomaterial after a predetermined period of time has elapsed. Therefore, in order to smoothly culture the biomaterial, the culture medium should be replaced at a predetermined period or the biomaterial should be moved to a new culture dish.

As the related art of supplying a new culture medium to the biomaterial, there are provided Patent Documents 1 and 2. In Patent Document 1, a configuration of forming channels in first and second substrates 110 and 150 has been described, and in Patent Document 2, a groove portion bottom 4 through which a fluid moves in a second substrate 3 has been described.

However, in inventions disclosed in these Patent Documents, since substrates separated from each other may be coupled to each other to thereby be integrated, it may be difficult to form, culture, or analyze various biomaterials. For example, in inventions disclosed in these Patent Documents, an experiment may not be performed in a state in which different biomaterials are limited to a specific zone. Further, in inventions disclosed in these Patent Documents, since only one analysis sample may be sent in the channel, it is difficult to perform a variety of analyses, and the culture medium may remain in the channel as it is, such that precision of the analysis of the biomaterial is deteriorated.

### RELATED ART DOCUMENT

(Patent Document 1) KR2011-128658 A
(Patent Document 2) JP2002-243734 A

### SUMMARY OF THE INVENTION

An aspect of the present invention provides a cell chip capable of collectively analyzing various biomaterials.

Another aspect of the present invention provides a cell chip in which a substrate to which a biomaterial is attached and a substrate in which a culture medium is stored may be selectively coupled to and decoupled from each other so as to allow a plurality of different biomaterials to be formed, cultured, and analyzed.

Another aspect of the present invention provides a cell chip capable of observing a metabolism reaction of a biomaterial to a medicinal substance under conditions similar to human body conditions.

According to an aspect of the present invention, there is provided a cell chip including: a first substrate having biomaterials fixed thereto; a second substrate provided with one or more receiving spaces having a culture medium stored therein; and a circulation unit circulating the culture medium stored in the receiving spaces.

The first substrate may include one or more protrusions formed on one surface thereof and having the biomaterials fixed thereto.

Each of the receiving spaces may have a cross-sectional shape allowing one or more biomaterials to be received therein.

At least one of the first substrate and the second substrate may be provided with provided with an interval maintaining member maintaining an interval between the first substrate and the second substrate.

The receiving spaces may be provided in plural, the plurality of receiving spaces being arranged in the second substrate in a length direction or a width direction, and one receiving space may be connected to one or more other receiving spaces, among the plurality of receiving spaces.

The receiving spaces may be provided in plural, the plurality of receiving spaces being arranged in the second substrate in a length direction and a width direction, and one receiving space may be connected to one or more other receiving spaces, among the plurality of receiving spaces.

The first substrate and the second substrate may be selectively coupled to and decoupled from each other.

According to another aspect of the present invention, there is provided a cell chip including: a first substrate having biomaterials fixed thereto; a second substrate provided with one or more receiving spaces having a culture medium stored therein; a third substrate coupled to the second substrate and provided with one or more culture medium supplementing spaces connected to the receiving spaces; and a circulation unit circulating the culture medium stored in the receiving spaces and the culture medium supplementing spaces.

The first substrate may include one or more protrusions formed on one surface thereof and having the biomaterials fixed thereto.

Each of the receiving spaces may have a cross-sectional shape allowing one or more biomaterials to be received therein.

At least one of the first substrate and the second substrate may be provided with an interval maintaining member maintaining an interval between the first substrate and the second substrate.

The receiving spaces may be provided in plural, the plurality of receiving spaces being arranged in the second substrate in a length direction or a width direction, and one receiving space may be connected to one or more other receiving spaces, among the plurality of receiving spaces.

The receiving spaces may be provided in plural, the plurality of receiving spaces being arranged in the second substrate in a length direction and a width direction, and one receiving space may be connected to one or more other receiving spaces, among the plurality of receiving spaces.

The culture medium supplementing space may be connected to the one or more receiving spaces.

The third substrate may further include a filtering member removing foreign objects included in the culture medium.

The circulation unit may be disposed in the culture medium supplementing spaces.

The first substrate and the second substrate may be selectively coupled to and decoupled from each other.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an exploded perspective view of a cell chip according to an embodiment of the present invention;

FIG. 2 is a bottom perspective view of a first substrate shown in FIG. 1;

FIG. 3 is an assembled perspective view of the cell chip shown in FIG. 1;

FIG. 4 is a cross-sectional view taken along line A-A of the cell chip shown in FIG. 3;

FIG. 5 is an exploded perspective view of a cell chip according to another embodiment of the present invention;

FIG. 6 is a bottom perspective view of a first substrate shown in FIG. 5;

FIG. 7 is an assembled perspective view of the cell chip shown in FIG. 5;

FIG. 8 is a cross-sectional view taken along line B-B of the cell chip shown in FIG. 7;

FIGS. 9 and 10 are views showing different shapes of a second substrate for describing different forms of culture medium circulation structures;

FIG. 11 is an exploded perspective view of a cell chip according to another embodiment of the present invention;

FIG. 12 is an assembled perspective view of the cell chip shown in FIG. 11;

FIG. 13 is a cut-away perspective view showing a cross section taken along line C-C of the cell chip shown in FIG. 12; and

FIG. 14 is a cross-sectional view taken along line C-C in order to describe another form of the cell chip shown in FIG. 11.

### DETAILED DESCRIPTION OF THE EMBODIMENTS

Generally, a reaction experiment or a culture experiment of a biomaterial is performed in a stagnating culture medium. However, since this experimental environment is different from an actual bio-environment in which blood or other fluid moves continuously, reliability of an experimental result may be low.

In addition, when the culture medium stagnates, waste matter generated due to metabolism of a biomaterial is accumulated in the culture medium as it is to hinder smooth culturing or cause staining of the biomaterial. Therefore, in the case in which the biomaterial is cultured in the stagnating culture medium, there is inconvenience that the culture medium should be frequently replaced.

The present invention, the object of which is to solve the above-mentioned problem, may improve reliability of an experimental result by allowing an experimental environment or a culture environment of the biomaterial to be similar to a bio-environment.

Further, in the present invention, since a substrate to which the biomaterial is attached and a substrate in which the culture medium is stored may be attached to or detached from each other a plurality of different biomaterials may be effectively repeatedly formed, cultured, and analyzed. To this end, the present invention provides a cell chip in which the culture medium is circulated, whereby all of the above-mentioned limitations may be solved.

Hereinafter, embodiments of the present invention will be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like elements.

In addition, a substrate used in the present invention is a member used for experimentation on a biomaterial. Therefore, a material of the substrate is not particularly limited. Accordingly, a substrate to be described below may be formed of silicon, glass, metal, or a polymer. Here, a kind of polymer is not particularly limited, but may be, for example, polymethylmethacrylate (PMMA), polycarbonate (PC), polystyrene (PS), polypropylene, a cyclic olefin copolymer, polynorbonene, a styrene-butadiene copolymer (SBC), or acrylonitrile butadiene styrene.

In addition, a method of manufacturing the substrate is not particularly limited. For example, the substrate may be manufactured by a photo-resist process, an etching process, an injection molding process, or the like.

In addition, a biomaterial mentioned in the present specification refers to various materials. For example, the biomaterial mentioned in the present specification may be a nucleic acid arrangement such as ribo nucleic acid (RNA), deoxyribonucleic acid (DNA), or the like, peptides, proteins, lipids, organic or inorganic chemical molecules, virus particles, prokaryotic cells, organelles, or the like. In addition, the biomaterial is not limited to a cell of a human being, but may be used as a having a meaning including a cell of various animals or plants.

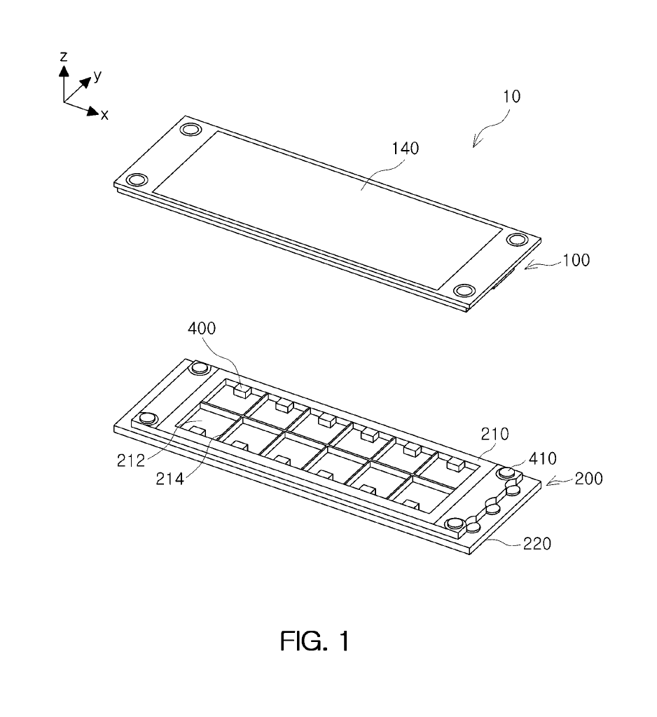

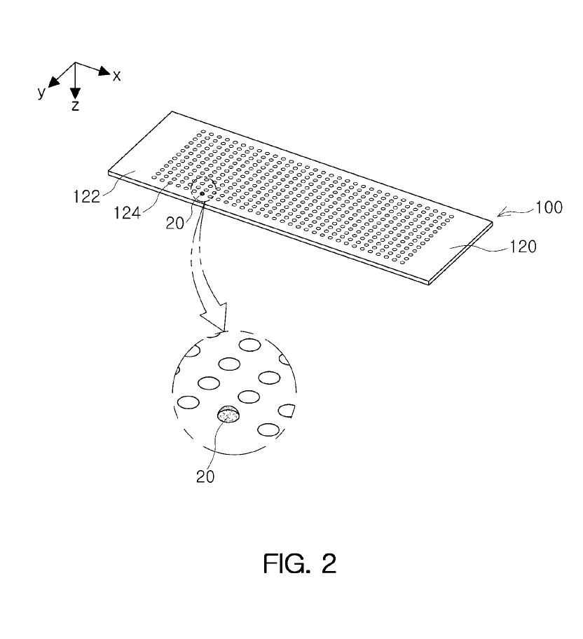

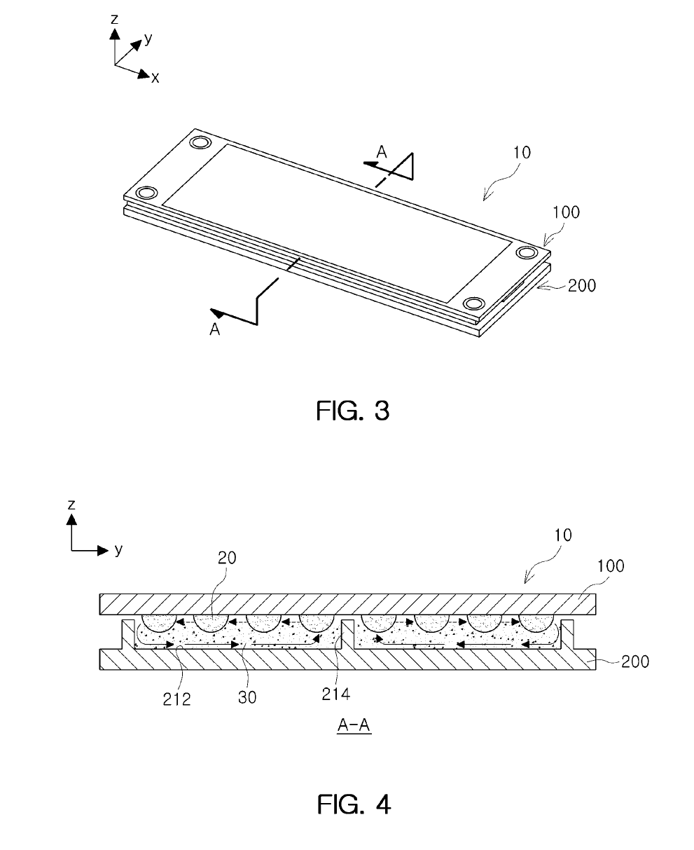

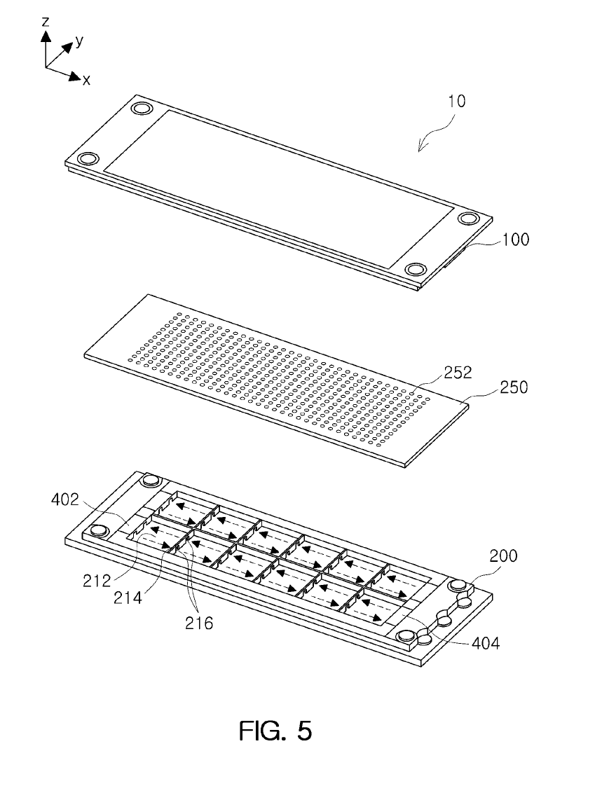

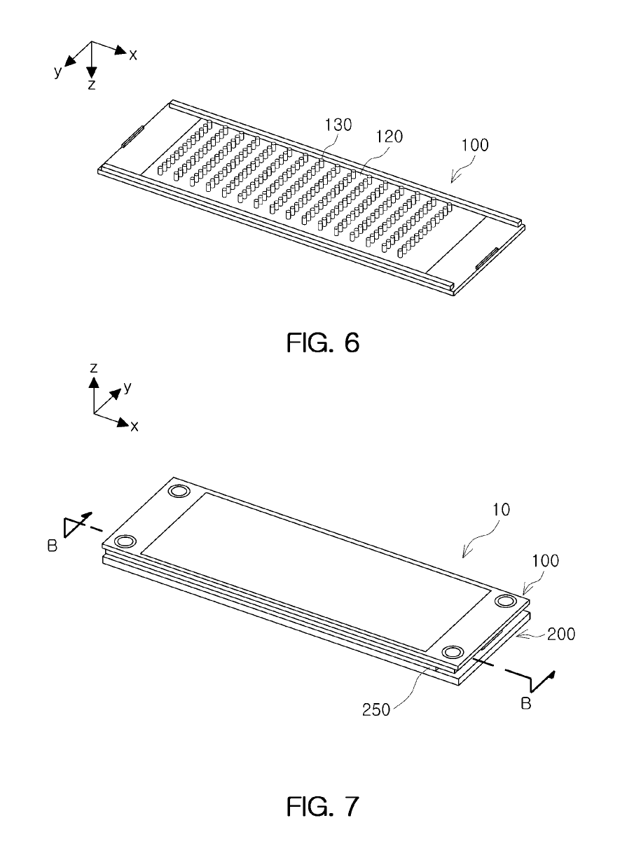

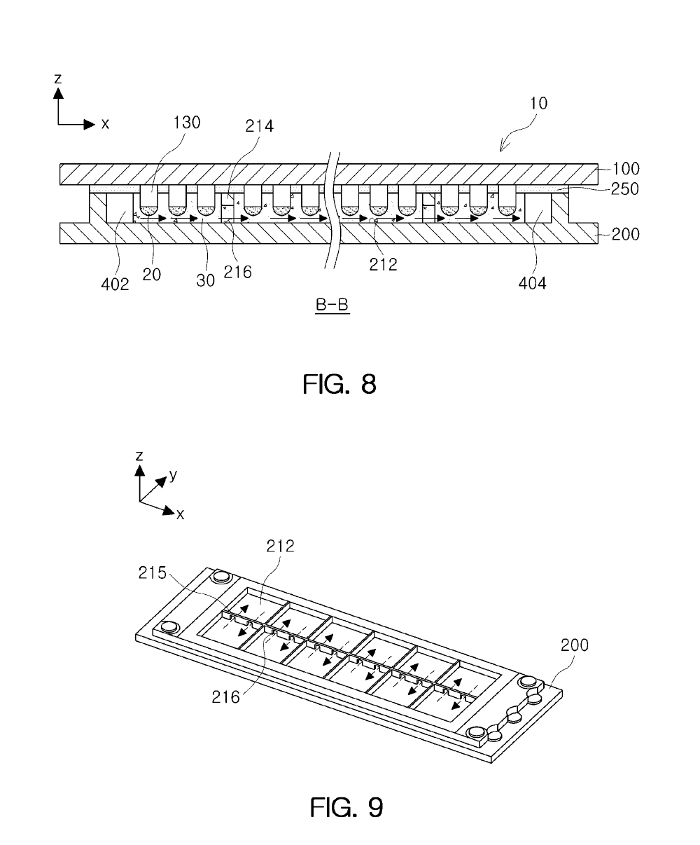

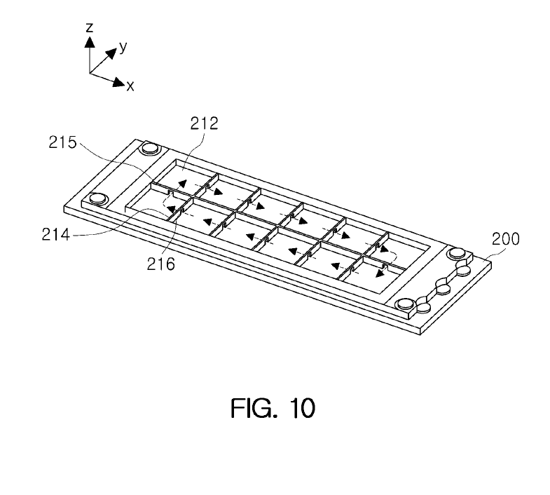

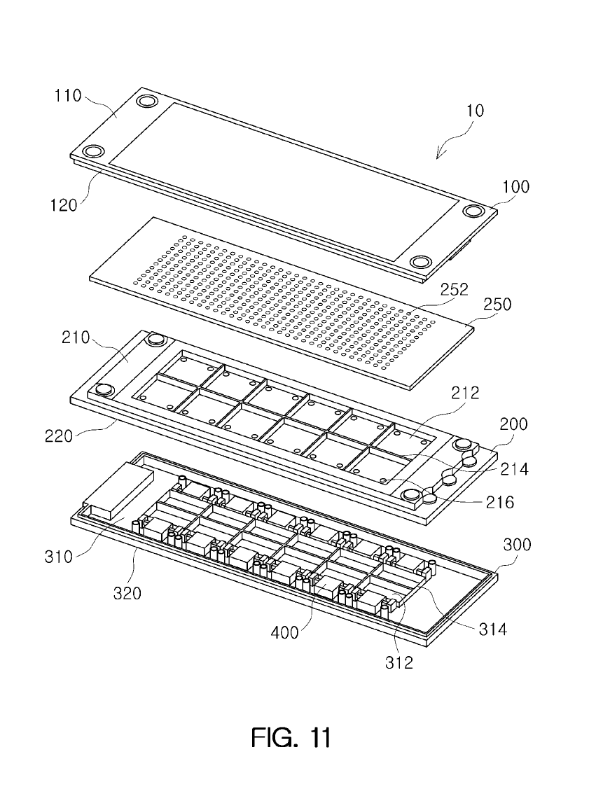

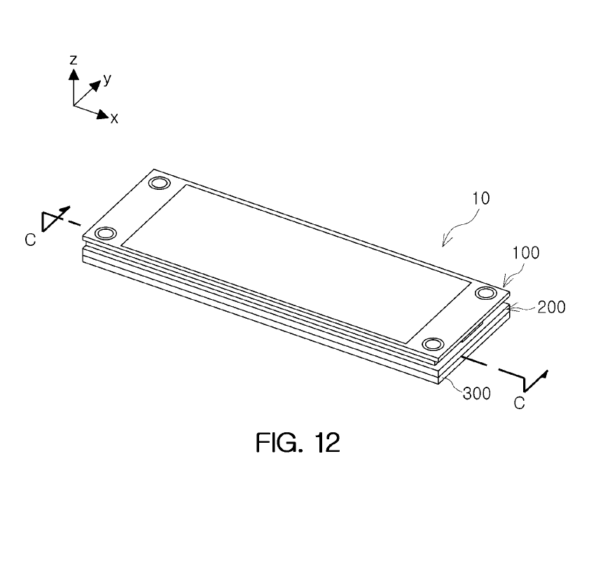

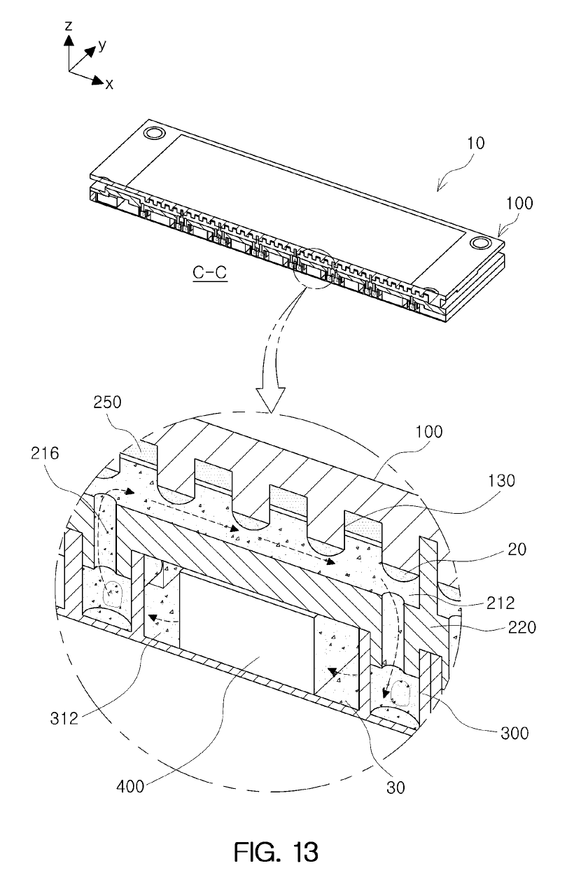

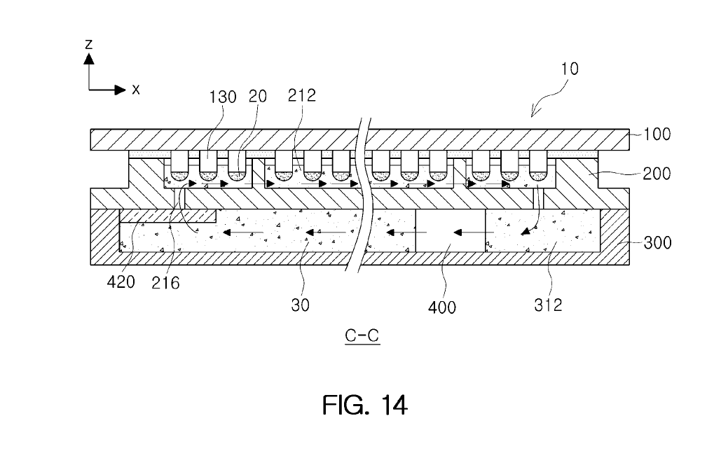

FIG. 1 is an exploded perspective view of a cell chip according to an embodiment of the present invention; FIG. 2 is a bottom perspective view of a first substrate shown in FIG. 1; FIG. 3 is an assembled perspective view of the cell chip shown in FIG. 1; FIG. 4 is a cross-sectional view taken along line A-A of the cell chip shown in FIG. 3; FIG. 5 is an exploded perspective view of a cell chip according to another embodiment of the present invention; FIG. 6 is a bottom perspective view of a first substrate shown in FIG. 5; FIG. 7 is an assembled perspective view of the cell chip shown in FIG. 5; FIG. 8 is a cross-sectional view taken along line B-B of the cell chip shown in FIG. 7; FIGS. 9 and 10 are views showing different shapes of a second substrate for describing different forms of culture medium circulation structures; FIG. 11 is an exploded perspective view of a cell chip according to another embodiment of the present invention; FIG. 12 is an assembled perspective view of the cell chip shown in FIG. 11; FIG. 13 is a cut-away perspective view showing a cross section taken along line C-C of the cell chip shown in FIG. 12; and FIG. 14 is a cross-sectional view taken along line C-C in order to describe another form of the cell chip shown in FIG. 11.

For reference, in the following specification, a first surface refers to an X-Y plane in a positive z-axis direction in FIG. 1, and a second surface refers to an X-Y plane in a negative z-axis direction in FIG. 1.

In addition, a first direction or a length direction refers to an X-axis direction in FIG. 1, and a second direction or a width direction refers to a Y-axis direction in FIG. 1.

A cell chip according to an embodiment of the present invention will be described with reference to FIGS. 1 through 4.

The cell chip 10 according to the present embodiment may include a first substrate 100 and a second substrate 200.

The first substrate 100 may generally have a plate shape lengthily extended in a single direction (the X-axis direction in FIG. 1), for example, a rectangular shape. However, the first substrate 100 is not limited to having the rectangular shape, but may also have other shapes.

The first substrate 100 may be formed of plastic. Since the first substrate 100 formed of plastic may be mass-produced through a mold, manufacturing costs may be decreased as compared with a biochip made of glass. In addition, since the first substrate 100 formed of plastic is relatively lighter and relatively more brittle than a substrate formed of glass, handling may be easy and a damage generation rate due to handling carelessness may be decreased.

The first substrate 100 may be used as a member to which biomaterials 20 are attached. To this end, at least one of first and second surfaces 110 and 120 of the first substrate 100 may be coated with a hydrophilic material. More specifically, one surface (the second surface 120 in the present embodiment) of the first substrate 100 may include a first region 122 coated with a hydrophobic material and second regions 124 coated with a hydrophilic material.

Here, the first region 122 may be a region to which the biomaterials 20 are not attached, and the second regions 124 may be regions to which the biomaterials 20 are attached. The second regions 124 may be formed at predetermined intervals in the first substrate 100 in a length direction (an X-axis direction in FIG. 2) and a width direction thereof (a Y-axis direction in FIG. 2).

The second substrate 200 may have a plate shape the same as or similar to that of the first substrate 100, as shown in FIG. 1. The second substrate 200 may be formed of plastic, similar to the first substrate 100.

The second substrate 200 may be used as a member in which a culture medium 30 is stored. To this end, the second substrate 200 may have one or more receiving spaces 212 in a first surface 210 thereof. The receiving spaces 212 may be formed at predetermined intervals in the second substrate 200 in a length direction or a width direction thereof or the length direction and the width direction thereof. Here, neighboring receiving spaces 212 may have a partition wall 214 formed therebetween in order to isolate the neighboring receiving spaces 212 from each other.

Each of the receiving spaces 212 may have a predetermined length L and a predetermined width W. Here, an area (L×W) of the receiving space 212 may be a size capable of receiving a plurality of biomaterials 20. That is, the receiving space 212 may receive all of the plurality of biomaterials 20 attached to the first substrate 100 in a state in which the first substrate 100 and the second substrate 200 are coupled to each other (See FIG. 4).

Meanwhile, the second substrate 200 may include an interval maintaining member 410 formed on the first surface 210 thereof. The interval maintaining member 410 may protrude from the first surface 210 of the second substrate 200 in the positive z-axis direction. The interval maintaining member 410 formed as described above may form a gap through which air may be introduced between the first substrate and the second substrate 100 and 200 in the state in which the first substrate 100 and the second substrate and 200 are coupled to each other.

A circulation unit 400 may be disposed on the second substrate 200. For example, the circulation unit 400 may be disposed on the first or second surface 210 or 220 of the second substrate 200. For reference, in the present embodiment, although the circulation unit 400 is disposed in each of the receiving spaces 212, it is not necessarily required to dispose the circulation unit 400 in the receiving space 212. For example, the circulation unit 400 may be positioned at an outer side of the receiving space 212.

The circulation unit 400 may circulate the culture medium 30. For example, the circulation unit 400 may apply pressure to the culture medium 30 such that the culture medium 30 continuously moves in the receiving space 212. To this end, the circulation unit 400 may include a small pump.

The cell chip 10 configured as described above may have a single chip shape as shown in FIG. 3 due to vertical coupling between the first substrate 100 and the second substrate 200. In the state in which the first substrate 100 and the second substrate 200 are coupled to each other, the biomaterials 20 of the first substrate 100 may be completely or partially immersed in the culture medium 30 of the second substrate 200, as shown in FIG. 4.

Hereinafter, a culture medium circulation structure of the cell chip 10 according to the present embodiment will be described.

The culture medium 30 may be stored in the receiving space 212 of the second substrate 200, and the circulation unit 400 may be disposed in the receiving space 212 of the second substrate 200. Here, the circulation unit 400 may continuously or periodically perform an operation of drawing or discharging the culture medium 30. Therefore, the culture medium 30 stored in the receiving space 212 may be circulated in a predetermined direction without stagnating in the receiving space 212. Here, the circulation of the culture medium 30 may be a circulation movement in a clockwise direction or a counterclockwise direction on a plane of the receiving space 212 (the X-Y plane in FIG. 1). Alternatively, the circulation-movement of the culture medium 30 may be circulation movement in a clockwise direction or a counterclockwise direction on a vertical plane of the receiving space 212 (an X-Z plane or a Y-Z plane in FIG. 1).

The circulation movement of the culture medium 30 as described above may activate a reaction between the biomaterials 20 and the culture medium 30 and make a culture environment of the biomaterials 20 similar to an actual human body environment. In addition, since the circulation movement of the culture medium 30 as described above removes a metabolite (for example, a waste product) generated in a metabolism process of the biomaterials 20 and supplies a new nutritive element to the biomaterials 20, a culture time of the biomaterials 20 may be extended. Therefore, in the cell chip 10 according to the present embodiment, the culture time of the biomaterials 20 in the culture medium 30 may be increased.

Further, in the cell chip 10 according to the present embodiment, since the first substrate 100 and the second substrate 200 may be coupled to and decoupled from each other, different biomaterials may be formed in an array on the first substrate 100. Here, the biomaterials may be formed on the first substrate in a discharging scheme using an inkjet or a contact scheme using a physical contact between the biomaterials and the first substrate. The first substrate 100 is re-coupled to another second substrate in which the same culture medium is stored, and then the same culture experiment or the same analyzing experiment may be repeatedly performed. Alternatively, the first substrate 100 is re-coupled to another second substrate in which a different culture medium is stored, and then different culture experiments or various analyzing experiments through various staining may be additionally performed.

A cell chip according to another embodiment of the present invention will be described with reference to FIGS. 5 through 10. For reference, in the present embodiment, components that are the same or similar to those of the embodiment of the present invention described above will be denoted by the same reference numerals, and a detailed description thereof will be omitted.

The cell chip 10 according to another embodiment of the present invention may include the first substrate 100, an intermediate substrate 250, and the second substrate 200. The cell chip 10 configured as described above may be different in terms of a shape of the first substrate 100 from the cell chip according to the aforementioned embodiment of the present invention. In addition, the cell chip 10 according to another embodiment of the present invention may be different from the cell chip according to the aforementioned embodiment of the present invention in that it further includes the intermediate substrate 250. In addition, the cell chip 10 according to another embodiment of the present invention may be different from a circulation structure of the culture medium 30 from the cell chip according to the aforementioned embodiment of the present invention.

Hereinafter, main components of the cell chip according to another embodiment of the present invention different from those of the cell chip according to the aforementioned embodiment of the present invention will be described.

The first substrate 100 may include a plurality of protrusions 130 as shown in FIG. 6. The protrusions 130 may be formed on the second surface 120 of the first substrate 100. Here, the protrusions 130 may protrude in a direction (that is, in the positive Z-axis direction) perpendicular to the second surface 120 of the first substrate 100. In addition, the protrusions 130 may be formed at predetermined intervals on the first substrate 100 in the length direction and the width direction thereof (that is, the X-axis direction and the Y-axis direction). All of the protrusions 130 formed as described above may have the same length and have one of a circular cross-sectional shape, a rectangular cross-sectional shape, and a polygonal cross-sectional shape. In addition, upper surfaces of the protrusions 130 may be processed such that the biomaterials are easily attached thereto or be coated with an auxiliary material (for example, a hydrophilic material) facilitating the attachment of the biomaterials.

The intermediate substrate 250 may be disposed between the first substrate 100 and the second substrate 200. More specifically, the intermediate substrate 250 may be disposed on the first surface 210 of the second substrate 200. The intermediate substrate 250 may be provided with holes 252 receiving the protrusions 130 of the first substrate 100 therein or penetrating therethrough. The holes 252 may be lengthily formed in a thickness direction of the intermediate substrate 250 (a Z-axis direction in FIG. 5) and correspond to the respective protrusions 130 of the first substrate 100. Therefore, in a state in which the first substrate 100, the intermediate substrate 250, and the second substrate 200 are coupled to one another, the protrusions 130 of the first substrate 100 may penetrate through the holes 252 of the intermediate substrate 250 and protrude toward receiving spaces 212 of the second substrate 200.

Meanwhile, the intermediate substrate 250, provided to block an introduction of external gas at the time of coupling between the first substrate 100 and the second substrate 200, may be omitted as needed.

The second substrate 200 may include a plurality of the receiving spaces 212 partitioned by partition walls 214 and 215. Here, a portion of the partition wall 214 may be provided with a connection path 216 connecting the receiving spaces 212 neighboring each other as shown in FIG. 5.

Circulation units 402 and 404 may be disposed on both distal ends of the second substrate 200. Here, the circulation unit 402 disposed at the left of the second substrate 200 may pump the culture medium 30 to the right, and the circulation unit 404 disposed at the right of the second substrate 200 may pump the culture medium 30 to the left. However, dispositional forms of the circulation units 402 and 404 are not limited thereto, but may be changed according to a circulation form of the culture medium 30.

The cell chip 10 configured as described above may be configured by coupling the first substrate 100, the intermediate substrate 250, and the second substrate 200, and may have a single chip shape as shown in FIG. 7.

Hereinafter, a culture medium circulation structure of the cell chip 10 according to the present embodiment will be described.

In the present embodiment, the culture medium 30 may be circulated in a single circulation unit including the plurality of receiving spaces 212. To this end, the partition walls 214 and 215 of the second substrate 200 may be provided with at least one connection path 216.

Meanwhile, in the case in which the receiving spaces 212 disposed in the length direction of the second substrate 200 are connected to each other as a single circulation space, the connection path 216 may be formed in the partition wall 214 in the width direction of the second substrate 200, as shown in FIG. 5.

Unlike this, in the case in which the receiving spaces 212 disposed in the width direction of the second substrate 200 are connected to each other as a single circulation space, the connection path 216 may be formed in the partition wall 215 in the length direction of the second substrate 200, as shown in FIG. 9. In this case, a circulation unit (not shown) may be mounted on the partition wall 215 in the length direction. However, a mounting position of the circulation unit is not limited thereto, but may be changed within a range of enabling a circulation flow of FIG. 5.

In addition, in the case in which all of the receiving spaces 212 of the second substrate 200 are connected to each other as a single circulation space, connection paths 216 may be formed in both of the partition walls 214 and 215, as shown in FIG. 10. In this case, a circulation unit (not shown) may be mounted at a plurality of partition walls 214. However, a mounting position of the circulation unit is not limited thereto, but may be changed within a range of enabling a circulation flow of FIG. 5.

In the cell chip 10 configured as described above, since the culture medium 30 is circulated in a unit of the plurality of receiving spaces, the number of circulation units 402 and 404 may be significantly decreased.

Further, in the cell chip 10 according to the present embodiment, since the culture medium 30 is circulated in a unit of the plurality of receiving spaces 212, different biomaterials 20 may be formed on the first substrate 100, whereby an experiment on a reaction between the biomaterials 20, in addition to a reaction between the culture medium 30 and the biomaterials 20, may be performed. Therefore, the present embodiment is advantageous for performing an experiment on an interaction between different biomaterials 20.

A cell chip according to another embodiment of the present invention will be described with reference to FIGS. 11 through 14. For reference, in the present embodiment, components that are the same as or similar to those of the embodiments of the present invention described above will be denoted by the same reference numerals, and a detailed description thereof will be omitted.

The cell chip 10 according to another embodiment of the present invention may include the first substrate 100, the intermediate substrate 250, the second substrate 200, and a third substrate 300. The cell chip 10 configured as described above may be different from the cell chip according to the aforementioned embodiments of the present invention in that it further includes the third substrate 300.

The cell chip 10 according to another embodiment of the present invention may be formed by sequentially coupling the first substrate 100, the intermediate substrate 250, the second substrate 200, and the third substrate 300 to one another. However, the intermediate substrate 250 may be omitted as needed.

The first substrate 100 may have the biomaterials attached to the second surface 120 thereof. The first substrate 100 may include the plurality of protrusions 130 (See FIG. 13) formed on the second surface 120 thereof so as to allow the biomaterials to be easily attached thereto. However, as needed, the second surface 120 of the first substrate 100 may include a region coated with a hydrophilic material and regions coated with a hydrophobic material, as shown in FIG. 2.

The second substrate 200 may include the receiving spaces 212 in which the culture medium 30 is stored. The receiving spaces 212 may be formed in the first surface 210 of the second substrate 200 and may be a plurality of spaces divided by the partition wall 214. The second surface 220 of the second substrate 200 may be provided with the connection path 216 connected to each of the receiving spaces 212. The connection path 216 may be lengthily formed in the second substrate 200 in the thickness direction (the Z-axis direction) and connect each receiving space 212 and a culture medium supplementing space 312 of the third substrate 300 to each other. Meanwhile, two or more connection paths 216 may be provided in each receiving space 212.

The third substrate 300 may include the culture medium supplementing space 312. The culture medium supplementing space 312 may be formed in a first surface 310 of the third substrate 300 and be divided into a plurality of spaces by a partition wall 314. Here, the number of culture medium supplementing spaces 312 divided by the partition wall 314 may be the same as the number of receiving spaces 212. However, as needed, the number of culture medium supplementing spaces 312 may be smaller than that of receiving spaces 212.

The circulation unit 400 may be disposed on the third substrate 300. For example, the circulation unit 400 may be disposed on the first or second surface 310 or 320 of the third substrate 300. Alternatively, the circulation unit 400 may be disposed in the culture medium supplementing space 312, as shown in FIG. 11. The circulation unit 400 disposed as described above may pump the culture medium stored in the culture medium supplementing space 312 to the receiving space 212 of the second substrate 200.

The cell chip 10 configured as described above may be configured as a single chip shown in FIG. 12 by sequentially vertically coupling the first substrate 100, the second substrate 200, and the third substrate 300 to one another (for reference, the intermediate substrate 250 may be added or omitted as needed).

Hereinafter, a coupling structure of the cell chip 10 configured as described above will be described.

The cell chip 10 according to another embodiment of the present invention may have a structure in which the first substrate 100, the second substrate 200, and the third substrate 300 are vertically coupled to one another, as shown in FIG. 13.

In a state in which the first substrate 100, the second substrate 200, and the third substrate 300 are coupled to one another, the protrusions 130 may be received in the receiving spaces 212 of the second substrate 200. In addition, each receiving space 212 of the second substrate 200 and the culture medium supplementing space 312 of the third substrate 300 may be connected to each other by the connection path 216. Here, the circulation unit 400 disposed on the third substrate 300 may pump the culture medium 30 in one direction to form a flow of the culture medium 30 circulated in the culture medium supplementing space 312 and the receiving space 212 (See FIG. 13).

Meanwhile, the culture medium 30 may also be circulated in such a manner that the culture medium 30 entirely passes through the cell chip 10 in the length direction thereof, as shown in FIG. 14.

Additionally, a filtering member 420 may be attached to the connection path 216 of the second substrate 200 to remove contaminants contained in the culture medium 30.

As set forth above, according to the embodiments of the present invention, the culture medium is circulated, whereby a nutritive element and a medicinal substance may be continuously supplied to the biomaterials.

In addition, according to the embodiment of the present invention, since the substrates configuring the cell chip may be coupled to and decoupled from each other, different biomaterials may be repeatedly formed, cultured, and analyzed.

Further, according to the embodiments of the present invention, since a culture environment of the biomaterials similar to a human body environment in which blood is circulated may be created, reliability in a metabolism reaction result on the biomaterials may be improved.

Furthermore, according to the embodiments of the present invention, since the culture medium is continuously circulated, a culture period of the biomaterials may increase.

While the present invention has been shown and described in connection with the embodiments, it will be apparent to those skilled in the art that modifications and variations can be made without departing from the spirit and scope of the invention as defined by the appended claims.

What is claimed is:

1. A cell chip comprising:
a first substrate having biomaterials fixed thereto;
a second substrate provided with one or more receiving spaces having a culture medium stored therein; and
a circulation unit circulating the culture medium stored in the receiving spaces.

2. The cell chip of claim 1, wherein the first substrate includes one or more protrusions formed on one surface thereof and having the biomaterials fixed thereto.

3. The cell chip of claim 1, wherein each of the receiving spaces has a cross-sectional shape allowing one or more biomaterials to be received therein.

4. The cell chip of claim 1, wherein at least one of the first substrate and the second substrate is provided with an interval maintaining member maintaining an interval between the first substrate and the second substrate.

5. The cell chip of claim 1, wherein the receiving spaces are provided in plural, the plurality of receiving spaces being arranged in the second substrate in a length direction or a width direction, and
one receiving space is connected to one or more other receiving spaces, among the plurality of receiving spaces.

6. The cell chip of claim 1, wherein the receiving spaces are provided in plural, the plurality of receiving spaces being arranged in the second substrate in a length direction and a width direction, and
one receiving space is connected to one or more other receiving spaces, among the plurality of receiving spaces.

7. The cell chip of claim 1, wherein the first substrate and the second substrate are selectively coupled to and decoupled from each other.

8. A cell chip comprising:
a first substrate having biomaterials fixed thereto;
a second substrate provided with one or more receiving spaces having a culture medium stored therein;
a third substrate coupled to the second substrate and provided with one or more culture medium supplementing spaces connected to the receiving spaces; and
a circulation unit circulating the culture medium stored in the receiving spaces and the culture medium supplementing spaces.

9. The cell chip of claim 8, wherein the first substrate includes one or more protrusions formed on one surface thereof and having the biomaterials fixed thereto.

10. The cell chip of claim 8, wherein each of the receiving spaces has a cross-sectional shape allowing one or more biomaterials to be received therein.

11. The cell chip of claim 8, wherein at least one of the first substrate and the second substrate is provided with an interval maintaining member maintaining an interval between the first substrate and the second substrate.

12. The cell chip of claim 8, wherein the receiving spaces are provided in plural, the plurality of receiving spaces being arranged in the second substrate in a length direction or a width direction, and
one receiving space is connected to one or more other receiving spaces, among the plurality of receiving spaces.

13. The cell chip of claim 8, wherein the receiving spaces are provided in plural, the plurality of receiving spaces being arranged in the second substrate in a length direction and a width direction, and
one receiving space is connected to one or more other receiving spaces, among the plurality of receiving spaces.

14. The cell chip of claim 8, wherein the culture medium supplementing spaces are connected to the one or more receiving spaces.

15. The cell chip of claim 8, wherein the third substrate further includes a filtering member removing foreign objects included in the culture medium.

16. The cell chip of claim 8, wherein the circulation unit is disposed in the culture medium supplementing spaces.

17. The cell chip of claim 8, wherein the first substrate and the second substrate are selectively coupled to and decoupled from each other.
